(19) United States
(12) Patent Application Publication
(10) Pub. No.: US 2008/0248091 A1
(43) Pub. Date: Oct. 9, 2008

(54) Charge Gradient Inhibition of Trypanosoma Cruzi and Other Parasite's Invasive Action

(76) Inventors: Tarynn M. Witten, Richmond, VA (US); Patricio A. Manque, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/570,022

(22) PCT Filed: Jun. 3, 2005
(86) PCT No.: PCT/US05/19257

§ 371 Date: Jan. 24, 2008

Related U.S. Application Data
(60) Provisional application No. 60/576,630, filed on Jun. 4, 2004.

(51) Int. Cl. A61K 9/127; A61K 9/14; A61P 33/00

(52) U.S. Cl. 424/450; 424/489

(57) ABSTRACT

Methods for delaying and/or preventing the binding of parasites to host cells (especially mammalian host cells) are provided. The methods delay or prevent infection of host cells and the development of disease symptoms. The methods delay or prevent the parasites from locating and binding to host cells, thereby reducing the infectivity of the parasite, and allow for adjuvant drug therapy to have a longer treatment time period before infection sets in. The methods involve modifying the charge field sensed by the parasite in the vicinity of potential host cells. Such modifications may be made by exposing the parasite and/or the host cell to charge-altering agents such as charged nanoparticles.

### BACKGROUND OF THE INVENTION

1. Field of the Invention

The invention generally relates to methods for delaying and/or preventing the binding of parasites to host cells, thereby preventing/delaying infection of the host cell and the subsequent development of disease symptoms. In particular, the invention provides methods to prevent/inhibit the binding of parasites to host cells by using therapies that modify the charge field sensed by the parasite in the vicinity of potential host cells.

2. Background of the Invention

Infectious diseases caused by parasites are one of the world's largest existing medical problems. There are many examples of parasitic diseases that cause debilitating and/or fatal consequences for humans, the most severe of which include malaria, schistosomiasis, and Chagas' disease. 41% of the world's population lives in areas where malaria is endemic, and it is estimated that between 7,000,000 to 2.7 million people die of the disease each year, 75% of whom are African children. It is estimated that approximately 2 billion people harbor schistosomiasis worms. Morbidity estimates are that 300 million individuals are severely ill with worms, of which 50% are school-age children, and mortality estimates for Africa alone find that the death toll due to schistosomiasis may be as high as 200,000 per year. In addition, millions of people, particularly in Latin America, are at risk for infection with the Trypanosoma cruzi (T. cruzi) parasite, the causal agent in Chagas' disease. In terms of disability adjusted life years, Chagas' disease is globally ranked behind only malaria and schistosomiasis as the most serious parasitic diseases worldwide. The cornerstone of any approach to effectively combating these parasitic diseases is the development of improved methods of disease prevention and treatment, including discovery of agents or therapies effective against the parasites, vaccines, etc. While some progress has been made in this area, current technology is focused mainly on vaccine development, and is not dealing with clinical treatment of parasitic diseases, particularly once a host is exposed to or infected by the parasite. Thus, there exists an ongoing need to identify effective methods to treat and/or prevent the development of symptoms of parasitic diseases, particularly after a host has already been exposed to or infected by the parasite.

### SUMMARY OF THE INVENTION

The present invention provides methods and compositions for delaying and/or preventing the binding of parasites to their targeted host cells, particularly after the host is exposed to or infected by the parasite.

It is an object of this invention to provide a method of delaying or preventing a parasite from locating and binding to a mammalian host cell. The method comprises the step of modifying a charge field sensed by the parasite in the vicinity of the host cell. In a preferred embodiment, the step of modification is carried out by exposing the parasite or the host cell or both to a charge-modulating agent, examples of which include charged nanoparticles (e.g. negatively charged nanoparticles) and charged liposomes. In a preferred embodiment of the method, the parasite is Trypanosoma cruzi and the mammalian host cells are muscle cells or neural cells.

The invention further provides a method of attenuating (e.g. preventing, delaying, or lessening) symptoms of disease caused by a parasite in a host. The method comprises the step of administering to the host a charge-modulating agent in an amount sufficient to prevent infection of the host by the parasite, delay infection of the host by the parasite, or decrease the number of parasites that infect the host. In a preferred embodiment, the parasite is T. cruzi and the host is a mammal. In preferred embodiments, the charge-modulating agent is a charged nanoparticle or a liposome. Preferably, the charged nanoparticles are negatively charged nanoparticles that bind to the parasite.

### BRIEF DESCRIPTION OF THE DRAWINGS

FIG. 1. Schematic representation of the life cycle of the T. cruzi parasite.

FIG. 2. Initialization of T. cruzi velocities.

FIG. 3. Illustration of directional cone for a given parasite.

FIG. 4. Illustration of forces on a spherical T. cruzi parasite.

FIG. 5. Illustration of hypothetical T. cruzi swimming function.

FIG. 6. Initialization of T. cruzi velocities.

FIG. 7. Initial construction figure.

FIG. 8. Illustration of the rotation sets for each for the angles.

FIG. 9. Illustration of the azimuthal rotation sets.

FIG. 10. Illustration of the azimuthal rotation sets.

FIG. 11. Illustration of the orbital rotation sets.

FIG. 12. Illustration of a hypothetical T. cruzi swimming function.

FIG. 13A and B. A, graphical depiction of total surface area of charged nanoparticle vs total surface area of nanoparticle; B, graphical depiction of ln of total surface area of charged nanoparticle vs ln of total surface area of nanoparticle.

FIG. 14 A-C. A, graphical depiction of total surface charge area for groups on nanoparticle vs total surface area; B, In total surface charge area for groups on nanoparticle vs ln total surface charge area; C, ratio of surface areas of charges to total nanoparticle surface areas vs ratio of surface areas.

FIGS. 15A and B. Percent of infected cells for particles N1-N5. A, G strain of T. cruzi; B, CL strain of T. cruzi.

FIG. 16. Graphical representation of percent of infected cells (G strain of T. cruzi) vs total charge.

FIG. 17. Graphical representation of percent of infected cells (G strain of T. cruzi) vs nanoparticle diameter.

FIG. 18. T. cruzi trypomastigotes with bound nanoparticles

### DETAILED DESCRIPTION OF THE PREFERRED EMBODIMENTS OF THE INVENTION

The present invention provides methods and compositions for inhibiting the binding of parasites to their host cells, thereby delaying and/or preventing the development of disease symptoms caused by the parasites. The invention is based on the surprising discovery that parasites sense their environment through charge-charge interactions, i.e. standard “repulsion-attraction” based upon charge. Thus, a parasite's ability to locate and gain entry into suitable host cells is governed by their ability to sense and respond to charge fields or gradients surrounding the parasite and the targeted host cell. By altering the charge environment of the parasite, the host cell, or both, the host-parasite dynamics are altered, and the parasite's ability to correctly identify, bind to and infect suitable host cells is attenuated or abolished. The methods delay or prevent the parasites from locating and binding to host cells, thereby reducing the infectivity of the parasite, and allow for adjuvant drug therapy to have a longer treatment time period before infection sets in.

In a preferred embodiment of the invention, the parasite whose infectivity is inhibited is Trypanosoma cruzi (T. cruzi) and the targeted host cells are, for example, nucleated mammalian cells such as muscle cells (e.g. cardiac and smooth muscle cells). Much of the discussion below and in the Examples section deals with T. cruzi. However, those of skill in the art will recognize that the methods of the present invention apply equally well to other parasite-host systems, examples of which include but are not limited to Entamobae hisotlytica, Cryptosporidium parvum and Plasmodium falciforum. However, since the physics principal behind the invention is independent of the organisms involved, the method generalizes to any host-parasite system in which the host and parasite co-exist (during the preinfective and infective stages) in a fluid environment such as the blood, lymph, or cerebrospinal system. In addition, as the mathematical physics is independent of the mechanism of parasitic motion, the method also applies to parasites that are flagellar, amoeboid or simply free moving.

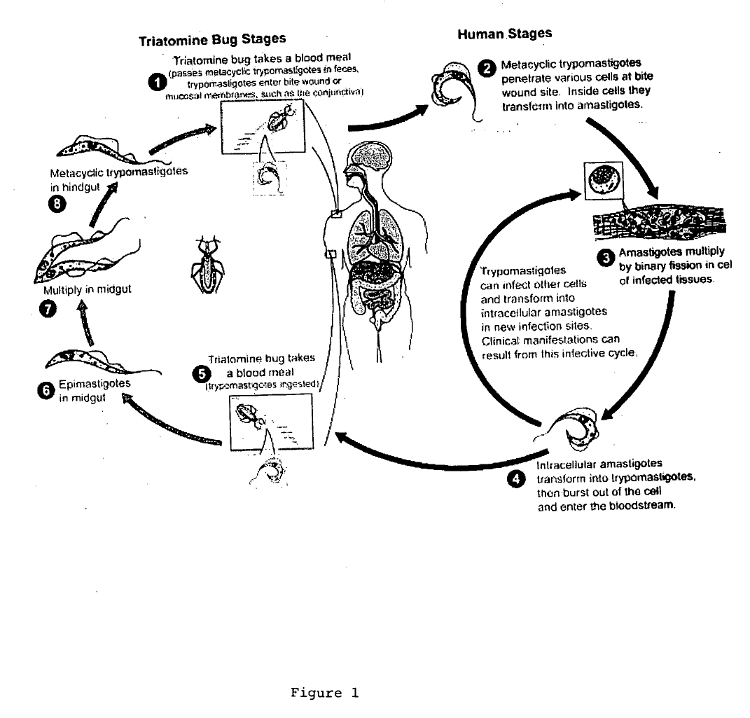

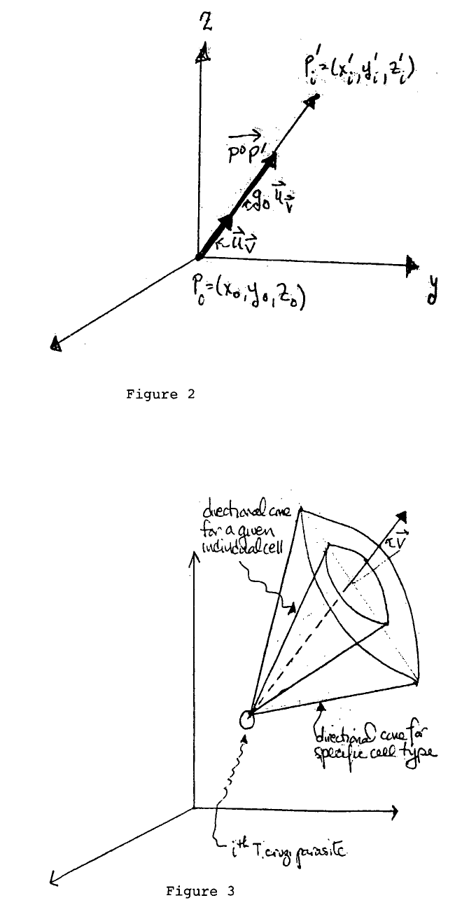

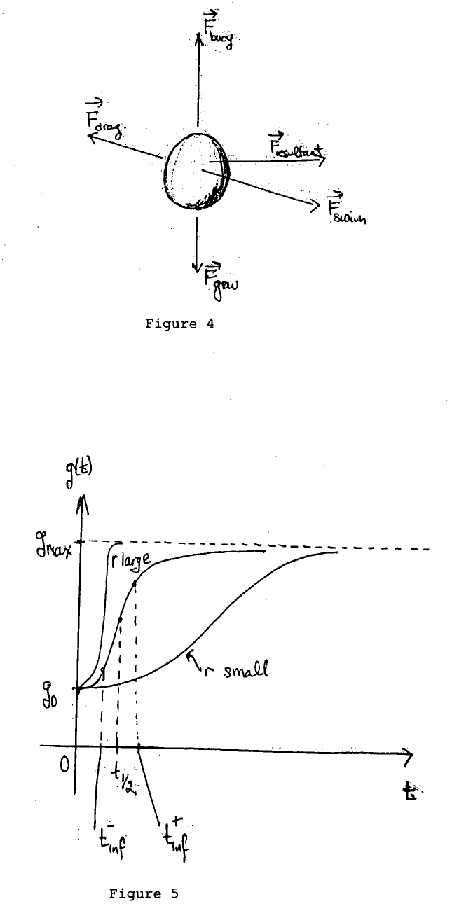

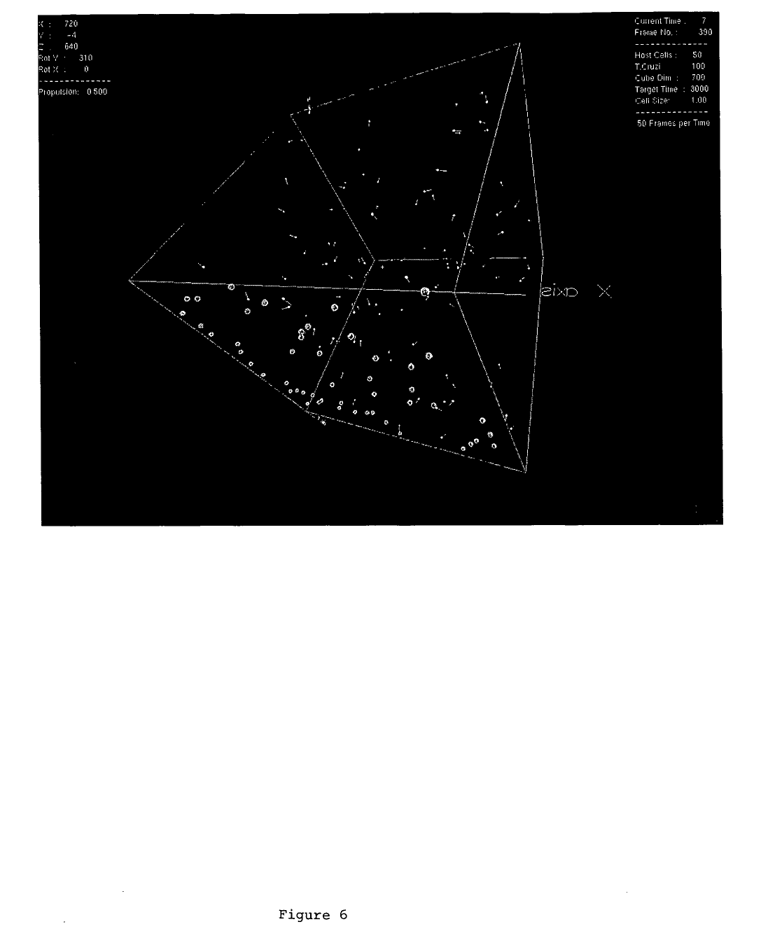

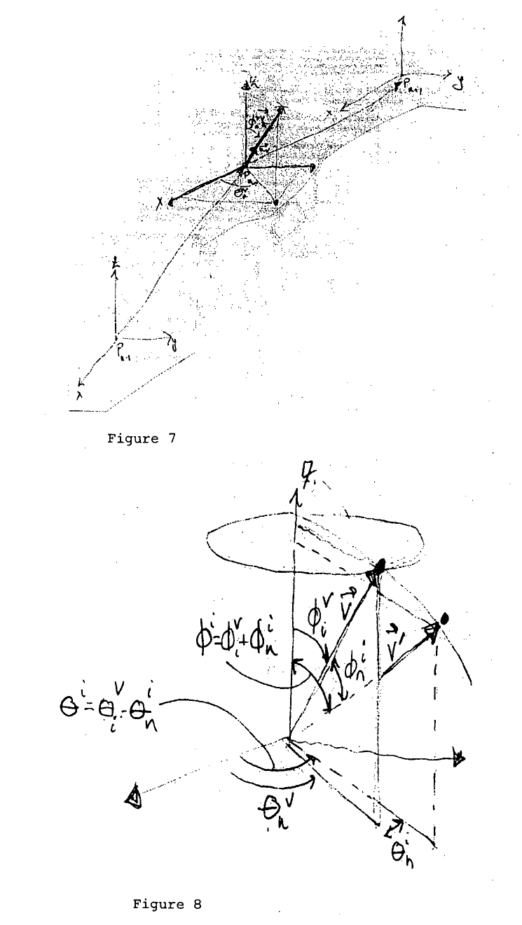

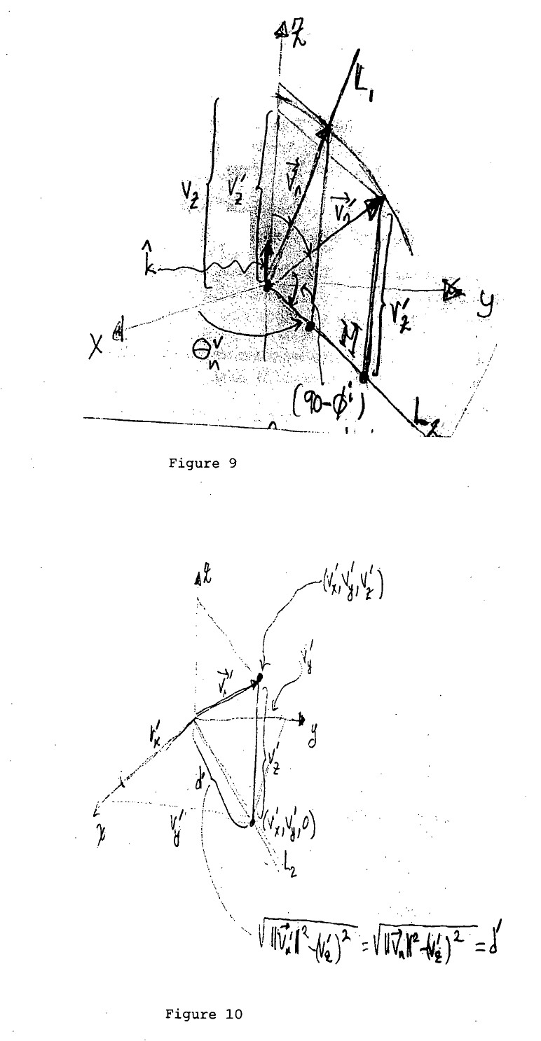

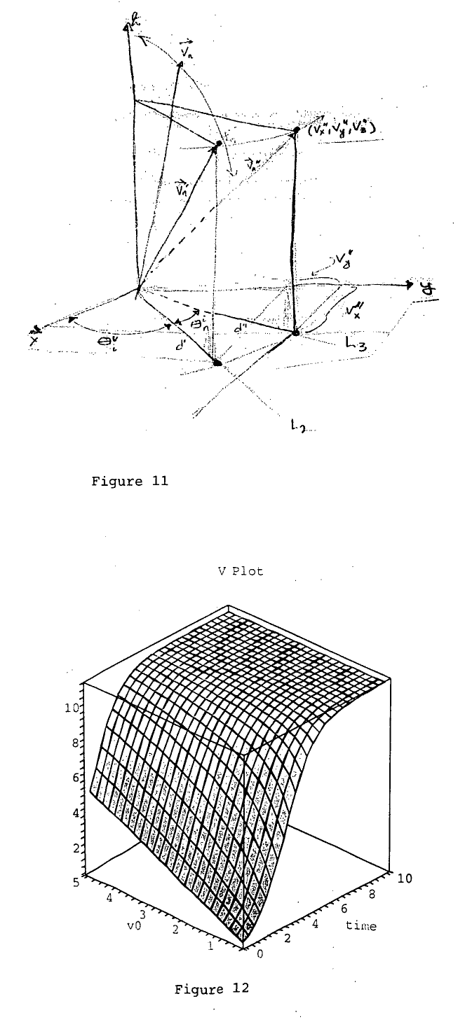

The life cycle of the exemplary parasite Trypanosoma cruzi is illustrated in FIG. 1. T. cruzi is transmitted from the Triatomine reduviid bug insect vector to a mammalian host via the feces of the insect, which is deposited on the skin of the host after the insect takes a blood meal. Motile metacyclic trypomastigote forms of the parasite are deposited with the feces and typically enter the host via skin lesions (e.g. by scratching the reduviid bug bite) and/or mucous membranes (e.g. by rubbing the eyes with contaminated fingers). Trypomastigotes may invade cells located in the areas of penetration (e.g. skin or mucosa) or circulate in the bloodstream of the host for a relatively short period of time and subsequently invade individual host cells. Inside the host cells, the parasites differentiate into small amastigote forms that replicate in the cytoplasm of the infected cells. Amastigotes subsequently differentiate back to the non-dividing motile trypomastigote form, emerge from the host cell, and re-invade new cells to propagate the infection within the host. The cycle in the insect vector is perpetuated by ingestion of the trypomastigote bloodstream form of the parasite by a reduviid bug (while feeding on the infected mammalian host), followed by differentiation, within the insect, to dividing epimastigote forms that subsequently differentiate into non-dividing trypomastigote forms, thus completing the parasite's life cycle.

Mammalian cell identification and invasion by T. cruzi is critical to its survival in the host, and thus to completion of its life cycle. Prior to the present invention, the mechanism used by the parasite to locate suitable host cells remained largely unknown, although some parasite molecules have been shown to be involved in the recognition of components of the extracellular matrix (Giordano R, Fouts D L, Tewari D, Colli W, Manning J E, ALves M J. Cloning of a surface membrane glycoprotein specific for the infective form of Trypanosoma cruzi having adhesive properties to laminin. J. Biol. Chem. 1999 Feb. 5; 274(6): 3461-8). While the mechanism of invasion of mammalian host cells by the parasite has not been fully elucidated, it is known to involve several parasite specific molecules, as well as host specific cell surface receptors and pathways (Burleigh B A, Woolsey A M. Cell signalling and Trypanosoma cruzi invasion. Cell Microbiol. 2002 November; 4(11): 701-11).

Without being bound by theory, the present invention is consistent with a mechanism of host cell-parasite interaction that is mediated by charge-charge interactions, i.e. identification and/or infection of the host cell by the infective form of the parasite is based on the parasite's ability to sense and respond to charge fields/gradients in its environment. The present invention provides methods and compositions to intervene in this process, and to thereby inhibit host cell-parasite interactions. This is accomplished by altering the charge environment in the vicinity of the host cell or parasite, or both, in order to interrupt such charge-charge interactions. By “in the vicinity of” we mean either at or near the surface of the host cell or parasite, or both, or in the environment that immediately surrounds the parasite and/or host cell, e.g. blood, lymph, saliva, and other body fluids. The method of the invention involves introducing into the environment of the parasite agents capable of interacting with charged species, e.g. on the surface of the parasite and/or host cell, and/or in the surrounding medium. Such agents alter the charge environment, for example, by altering the charge of the surface of the parasite itself, by altering the surface charge of the host cell, by altering the charge fields of the medium in which the infective form of the parasite and the host cell exist, (e.g. the solution that surrounds and/or is present between the parasite or the host cell, or both), or by any combination of these factors.

According to the invention, the charge environment of the parasite or host cell (or both) is altered by exposing the parasite or host cell (or both) to one or more agents capable of altering that environment. Such agents are characterized by 1) the ability to carry a predefined surface charge or volume charge density, and 2) the ability to reach the physical location of the parasite host targets without harming the host. Therefore, the agents need to be less than or equal to the size of the parasites. Examples of such charge-carrying agents include but are not limited to charged nanoparticles such as latex and charged organic agents such as liposomes, with the preferred agents being charged nanoparticles and charge-carrying liposomes. Such nanoparticles will typically be in the range of about 1 nanometer to about 10 microns in diameter. Those of skill in the art will recognize that carriers such as liposomes can by charge modified (e.g. by the attachment of antibodies or other charge-carrying agents).

The agents that are used in the practice of the present invention are typically administered to an individual or patient (e.g. a mammal) that has been exposed to or infected by a parasite, or who is at risk for such exposure/infection. The agents may be administered by a variety of means that are known to those of skill in the art, examples of which include but are not limited to oral or parenteral, including intravenously, intramuscularly, subcutaneously, etc., or by other routes (e.g. transdermal, sublingual, aerosol, etc.). The preferred method of administration is intravenous.

The agents can be administered in the pure form or in a pharmaceutically acceptable formulation including suitable elixirs, binders, and the like or as pharmaceutically acceptable salts or other derivatives. It should be understood that the pharmaceutically acceptable formulations and salts include liquid and solid materials conventionally utilized to prepare injectable dosage forms and solid dosage forms such as tablets and capsules. Water may be used for the preparation of injectable compositions which may also include conventional buffers and agents to render the injectable composition isotonic. Other potential additives include: colorants; surfactants (TWEEN, oleic acid, etc.); and binders or encapsulants (lactose, liposomes, etc). Solid diluents and excipients include lactose, starch, conventional disintegrating agents, coatings and the like. Preservatives such as methyl paraben or benzalkium chloride may also be used. Depending on the formulation, it is expected that the active agent will consist of 1-99% of the composition and the vehicular “carrier” will constitute 1-99% of the composition. The pharmaceutical compositions of the present invention may include any suitable pharmaceutically acceptable additives or adjuncts to the extent that they do not hinder or interfere with the therapeutic effect of the agent.

The agents may be administered to any mammal in need thereof, including humans, and other animals, e.g. domestic pets, animals that are raised commercially (e.g. cattle, horses, pigs, etc.), and the like.

Generally, for parenteral administration in humans, dosages in the range of from about 0.1 to about 1000 mg of active agent will be administered. The level of efficacy and optimal amount of dosage may vary from agent to agent, and the precise amount administered will vary from case to case, depending, for example, on the species being treated, and such factors as the size, weight, age, gender, general health, etc., of the patient.

The agents that are administered according to the methods of the present invention may be used either prophylactically to prevent initial infection of host cells by the parasite, or after infection to mitigate further infection. In the case of T. cruzi, the agents may be administered prophylactically (e.g. on a routine basis) prior to known exposure to and/or transmission of the parasite, or in response to a known exposure and/or transmission event. In the latter case, administration as soon as possible after transmission is preferred, since the agent can then act while the infective trypomastigotes are still in the bloodstream of the host, and thus help to prevent or to slow the entry of the trypomastigotes into host cells. However, administration in chronically infected individuals is also contemplated. Due to the cyclic nature of the T. cruzi life cycle, the trypomastigote form of the parasite appears and reappears many times throughout the lifetime of an infected individual. The methods of the present invention are useful for inhibiting the infectivity of the parasite during subsequent bouts of reinfection by this process as well, thus preventing or slowing the progression of Chagas' disease.

According to the invention, one or more charge-modulating agents is administered to an individual in need thereof. Thus, the invention contemplates the administration of either one or more than one of such agents at a time, either separately or in a mixture that contains more than one agent. In addition, the agents of the present invention may be used either alone or with other agents (e.g. drugs) that are used to treat parasitic infections. Examples of such agents/drugs include but are not limited to various vaccines, antibiotics, etc. The agents employed in the methods of the present invention may be administered separately from such drugs (i.e. in separate compositions). Alternatively, they may be co-administered together with other suitable drugs as a mixture, in a single composition.

Administration of charge-altering agents according to the methods of the present invention may be effective on a number of levels. For example, the agents may prevent the parasite from accurately sensing its surroundings and identifying suitable host cells, either partially or completely. In this case, some or all parasites in the blood stream may thus not even attempt to infect host cells. Alternatively (or in addition) some or all of the parasites may locate host cells and attempt but fail to bind to the host cell, or bind in a manner that does not result in entry into and thus infection of the host cell. In addition, the administration of the agents may prolong (i.e. slow down) successful binding and entry into a host cell. Infection of a host cell may be completely inhibited, or may simply be slowed as a result of the administration of the agents, i.e. fewer parasites may be infective, or the process of infection may be slowed, or both. The results are that no or fewer host cells are infected, and/or the process of infection is delayed. In the latter case, the amount of time during which the parasite is in the blood stream is increased. Thus, the parasite is advantageously exposed to other therapeutic agents (e.g. various drugs) or even to elements of the hosts own immune system for a longer time, allowing increased killing of the parasite, and a decrease in the infectivity of the parasite.

The methods of the invention may also be used to treat blood that is destined for use in transfusions.

### Example 2

### Blocking of Binding of T. cruzi Trypomastigotes to L6 Rat Muscle Cells Material and Methods

### Mammalian Cell Invasion Assay.

Trypomastigote forms derived from cultured cells of two different strains of T. cruzi (G and CL) were used in these experiments. Trypomastigotes were used because they are the bloodborne, infective form of T. cruzi. L6 (rat muscle) cells were grown to confluence at 37° C. in Dulbecco's minimum essential medium (DMEM) supplemented with 10% fetal calf serum (FCS), streptomycin (100 μg/ml) and penicillin (100 U/ml) in a humidified 5% CO₂ atmosphere. L6 muscle cells were utilized because they are a recognized in vitro model for determining parasite infectivity of mammalian cells.

The nanoparticles used in the study were negatively charged polystyrene micorspneres with sulphate functional groups on the surface. In such particles, the surface charge is pH independent and is stable at a wide range of pH. The surface of the particles is hydrophic in nature. Five types of such nanoparticles differing in mean diameter and charge density were used in the study (see Table 1). The nanoparticles were obtained from Dynamics Corporation, Oregon (internet address “ideclatex.com” or “probes.com”.

TABLE 1

| | | | | | Area | |
|---|---|---|---|---|---|---|
| | | Specific | | Surface | per | |
| | Mean | surface | Charge | charge | charge | Charge |
| | Diameter | area | content | density | group | groups per |
| Name | (μm) | (cm²/g) | (μEq/g) | (μC/cm²) | (Å/SO₄) | particle |
| 1 | 4.6 | 1.2E+04 | 1.9 | 14.7 | 109 | 6.1E+07 |
| 2 | 1.6 | 3.6E+04 | 3.4 | 9.2 | 174 | 4.6E+06 |
| 3 | 2.7 | 2.1E+04 | 2.3 | 10.5 | 152 | 1.5E+05 |
| 4 | 0.42 | 1.4E+05 | 4.6 | 3.3 | 488 | 1.1E+05 |
| 5 | 0.65 | 8.7E+04 | 7.8 | 8.6 | 187 | 7.1E+05 |

Mammalian cell invasion assays were carried out by seeding 1×10⁶ trypomastigote forms onto each well of 24-well plates containing 13-mm diameter round glass coverslip coated with 2×10⁵ L6 cells. Five different types of nanoparticles were tested, with one type added in each well and incubated for 1 hour. As a negative control, no nanoparticles were added to control wells. After incubation, the cells were washed 3 times using DMEM and incubated again in DMEM 10% FCS for 72 hrs at 37° C., 5% CO₂. The triplicate coverslips were then washed in phosphate buffered saline (PBS) and stained with Giemsa. The number of intracellular parasites was counted in 500 cells. All experiments were carried out in triplicate.

### Binding Assay.

Parasites (1×10⁵) were incubated in the presence or absence of the set of five nanoparticles for 1 hour at 28° C. After the incubation, the parasites were visualized using microscopy and the percent of parasites with attached nanoparticles was determined by counting 500 parasites.

### Results.

To quantify the effect of negatively-charged nanoparticles on the infectivity of Trypanosoma cruzi, a cell invasion assay was performed as described above in Materials and Methods. Five different types of nanoparticles were tested (see Table 1).

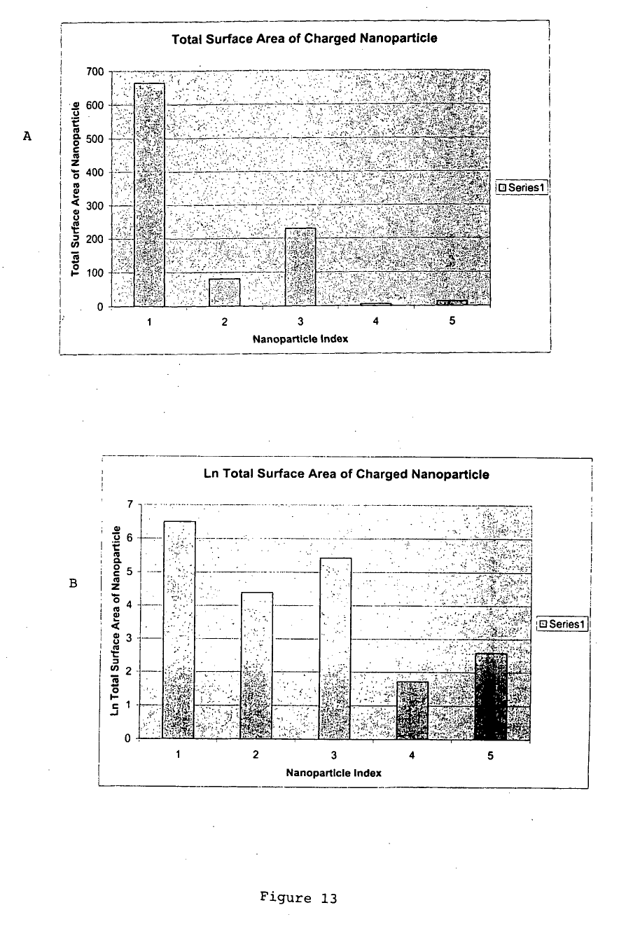

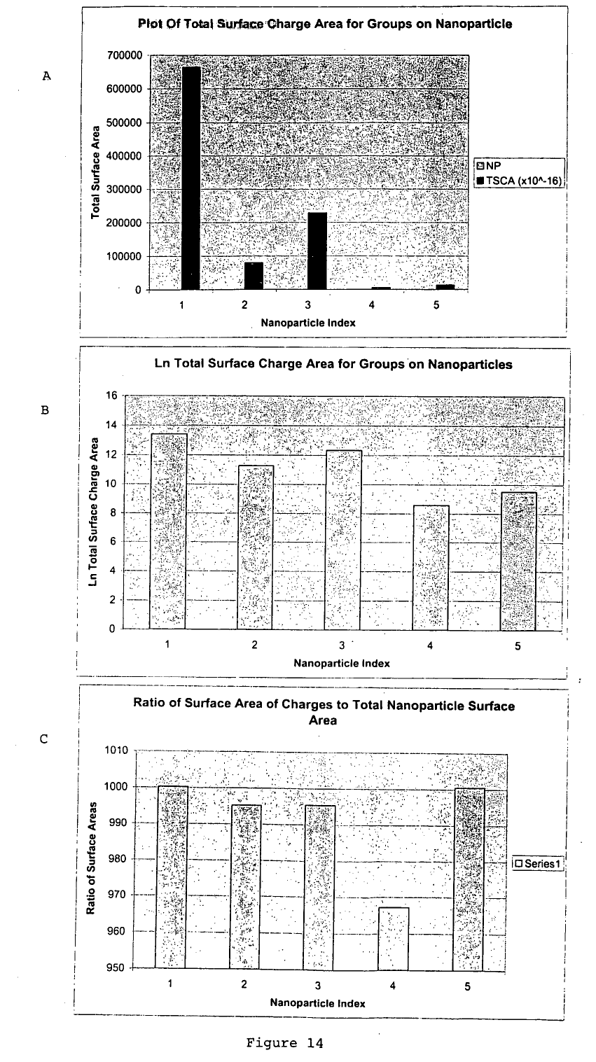

FIGS. 13 A and B shows the total surface area of the nanoparticles and the ln (natural log) of the surface area, respectively. FIG. 14A-C illustrates: the total surface charge area for the groups on the nanoparticles, the ln of the total surface area, and the ratios of surface area charges to the total nanoparticle surface area, respectively.

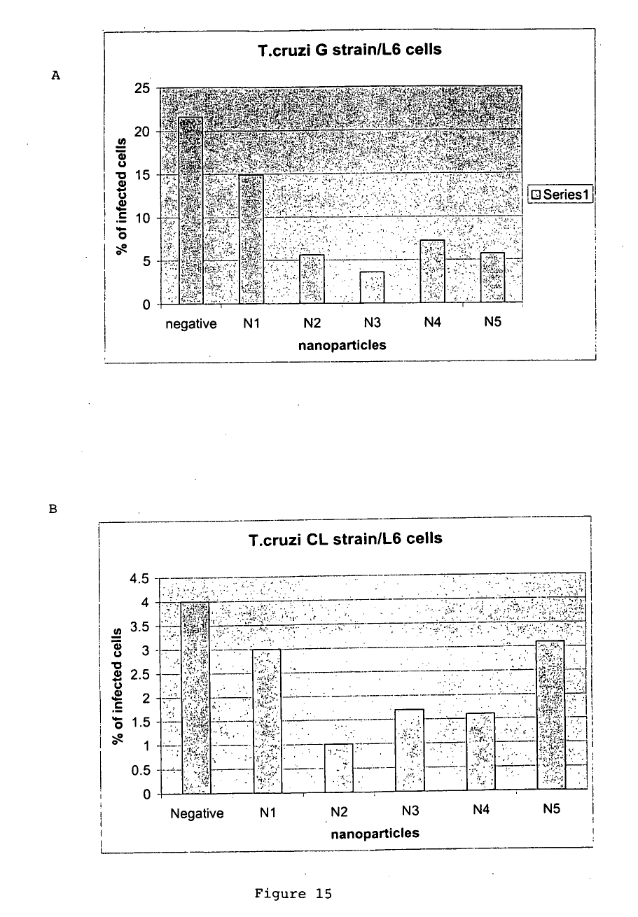

The results of the cell invasion assay with strains G and CL are presented in FIGS. 15A and B, respectively. As can be seen, the results show that each of the five types of nanoparticles inhibited the ability of T. cruzi trypomastigotes to infect mammalian muscle cells. The degree of inhibition varied from nanoparticle to nanoparticle, and was also dependent on the particular strain of T. cruzi that was inhibited. Interestingly, the largest inhibitory effect was observed in the CL strain, which is characterized as a highly infective strain.

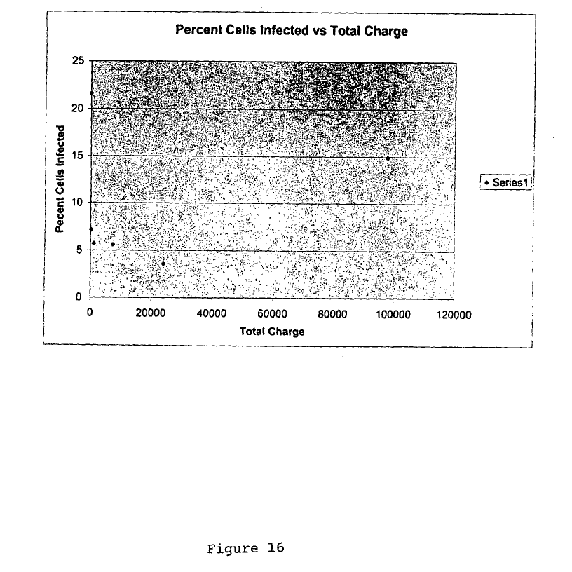

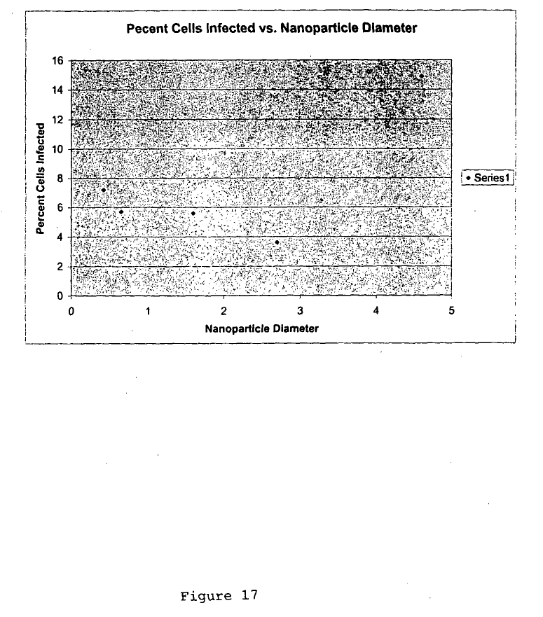

The data for T. cruzi strain G is presented in terms of the percent of cells infected vs total charge in FIG. 16, and as percent cells infected vs nanoparticle diameter in FIG. 17.

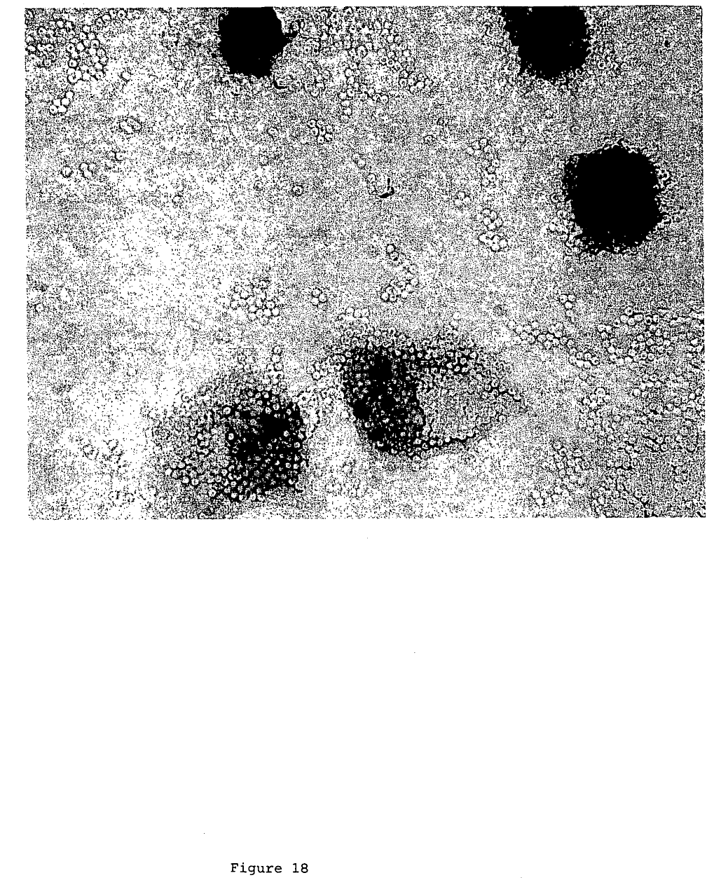

In order to determine whether the nanoparticles bind to the parasites and interfere with their interaction with host cells, a binding assay was performed using the five types of nanoparticles. The results, which are presented in Table 2, showed that all of the nanoparticles that could be visualized bound to the parasites, albeit with differing degrees of efficiency. A representative view of T. cruzi trypomastigotes with bound nanoparticles is shown in FIG. 18.

TABLE 2

| T. cruzi strain | Nanoparticle | % of parasites with attached nanoparticles |
|---|---|---|
| G | 1 | 58.86 |
| G | 2 | 79.07 |
| G | 3 | 69.36 |
| G | 4 | Indeterminate* |
| G | 5 | Indeterminate* |
| CL | 1 | 36.82 |
| CL | 2 | 78.22 |
| CL | 3 | 64.85 |
| CL | 4 | Indeterminate* |
| CL | 5 | Indeterminate* |

*Nanoparticle was too small to visualize by this method

The mathematical physics developed in the equations of Example 1 directly demonstrate that it is possible to make use of charge-charge interactions between the T. cruzi parasite and another object to either inhibit or stop the binding of the parasite to the host cell. Because of the general nature of the physics (i.e. not parasite or host cell-dependent), this methodology can be extended to any biological host pathogen system.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of delaying or preventing a parasite from locating and binding to a mammalian host cell, comprising the step of
modifying a charge field sensed by the parasite in the vicinity of the host cell.

2. The method of claim 1, wherein said step of modification is carried out by exposing said parasite or said host cell or both to a charge-modulating agent.

3. The method of claim 2, wherein said charge-modulating agent is selected from the group consisting of charged nanoparticles and liposomes.

4. The method of claim 3, wherein said charged nanoparticles are negatively charged nanoparticles.

5. The method of claim 1, wherein said parasite is Trypanosoma cruzi.

6. The method of claim 1, wherein said mammalian host cells are muscle cells or neural cells.

7. A method of attenuating symptoms of disease caused by a parasite in a host, comprising the step of
administering to said host a charge-modulating agent in an amount sufficient to prevent infection of said host by said parasite, delay infection of said host by said parasite, or decrease the number of parasites that infect said host.

8. The method of claim 7, wherein said parasite is T. cruzi and said host is a mammal.

9. The method of claim 7, wherein said charge-modulating agent is selected from the group consisting of charged nanoparticles and liposomes.

10. The method of claim 9, wherein said charged nanoparticles are negatively charged nanoparticles that bind to said parasite.

11. The method of claim 9, further comprising the step of administering a drug after said step of administering to said charge-modulating agent.
